(19) United States
(12) Patent Application Publication
(10) Pub. No.: US 2015/0073478 A1
(43) Pub. Date: Mar. 12, 2015

(54) SOFT TISSUE SUTURE ANCHOR

(71) Applicant: VasoStitch, Inc., Los Altos, CA (US)

(72) Inventors: Amir Belson, Los Altos, CA (US); Andy Denison, Temecula, CA (US); Eric Johnson, Temecula, CA (US); Phillip Burke, Pala, CA (US); David R. Lari, San Francisco, CA (US); Michael Schaller, Redwood City, CA (US); Luke Clauson, Redwood City, CA (US)

(21) Appl. No.: 14/541,495

(22) Filed: Nov. 14, 2014

Related U.S. Application Data
(63) Continuation of application No. PCT/US2013/040990, filed on May 14, 2013.
(60) Provisional application No. 61/646,735, filed on May 14, 2012.

(51) Int. Cl. A61B 17/04

(52) U.S. Cl. CPC A61B 17/0401; A61B 2017/0437; A61B 2017/0445; A61B 2017/0464
USPC 606/232

(57) ABSTRACT

Suture anchoring devices include retention features that will hold the anchoring devices in place and resist displacement when tension is applied to an attached suture in a direction opposite direction of placement. The anchors may be deployed by a needle shaft in a placement direction (which may be straight, curved, or a combination of both), and the needle shaft may have a sharpened or a blunt tip depending on the nature of the anchor. The anchors will find use when a distal end of the suture attached to the anchor is to be left in place in solid tissue so that it cannot be tied off.

### CROSS-REFERENCE TO RELATED APPLICATIONS

This application is a continuation of International Patent Application No. PCT/US2013/040990 (Attorney Docket No. 39277-707.601), filed May 14, 2013, which claims priority to Provisional Application No. 61/646,735 (Attorney Docket No. 39277-707.101), filed May 14, 2012, the entire contents of which are incorporated herein by reference.

### BACKGROUND OF THE INVENTION

Sutures are commonly used in surgery for incision and wound closure, with the primary function of the suture being to hold and join tissues together. Sutures are generally placed via a needle connected at a distal end of the suture. Typically, the needle penetrates the tissue and pulls the suture is through the desired tissues in any number of patterns, and the suture is knotted to seal the wound. The size, shape, and material of the suture may vary widely depending on the particular application. In instances where a knot is not ideal or simple to implement, alternatives such as barbed sutures may be used. Such sutures consist of retention features along the suture that provide a source of attachment and security to tissue. Depending on the application the barbed suture may or may not provide sufficient retention.

### SUMMARY OF THE INVENTION

The present invention provides anchoring devices which are useful with sutures to be placed in soft tissues. The sutures may be any type of surgical suture which may vary significantly in material properties, size, pattern, finish, and the like. The anchoring devices include retention features that will hold the anchor in place and resist displacement of an attached suture in a direction opposite direction of placement. That is, the anchors allow the suture to be advanced in a placement direction (which may be straight, curved, or a combination of both) but will prevent the suture from being drawn back in a direction opposite to the placement direction.

The anchors are particularly useful when the distal end of the suture (which carries the anchor) is to be left in place in solid tissue so that it cannot be tied off. Such sutures may be advanced on the distal end of a blunt or sharpened needle shaft in the placement direction and, once the distal end of the suture has reached a target location in the soft tissue, the needle shaft may be detached by drawing the shaft in a direction opposite to the placement direction, usually being back through the tissue tract that has been formed by advancement of the anchor and needle shaft. For convenience, the placement direction will usually be referred to as distal and the withdrawal direction will be referred to as proximal. After the needle shaft has been displaced, the suture will remain in place being anchored by the suture anchor.

A suture that has a distal anchoring device is useful for several reasons. The anchor can maintain the suture in place for procedures where the distal end of the suture is embedded in tissue or is placed in a cavity that is not easily accessible. Similar to conventional barbed suture, such a device provides the benefit of being self-anchoring (at the distal end) and aids with suture management as only the proximal end of the suture requires management. A distal anchoring device is further useful in that it may be used with, and its performance is not directly dependent on, most types of suture materials, sizes, finishes, braiding patterns, etc. Therefore, a device which securely anchors a suture in place without the need to tie the distal and proximal ends together would be useful.

In a specific embodiment, an anchor for attaching to a distal end of a length of suture comprises a tip body having a distal end and a proximal end. At least one retention element projects laterally from the tip body, where retention element is configured to permit the tip body to be advanced in a distal direction through tissue and to prevent the tip body from being withdrawn in a proximal direction from the tissue. A suture attachment element on the tip body allows a length of conventional surgical suture to be tied or otherwise attached to the anchor. A needle shaft engagement surface is formed on the tip body to permit a distal end of a needle shaft to engage and push to push the tip body in a distal direction through soft tissue. The surface is further configured disengage from the tip body when drawn in a proximal direction. Usually, the engagement and disengagement will be “passive” so that the engagement occurs when the needle shaft is pushed in a distal direction against the engagement surface and that disengagement occurs when the needle shaft is pulled back proximally from the engagement surface. In other instances, however, active mechanisms including latches, pull wires, screws, detents, and the like could be employed.

In one aspect, the tip body may have a tissue-penetrating distal end so that it may be used with the needle shaft having a blunt end. For example, the engagement surface of the tip body may include a pin surrounded by a shoulder at its proximal end, wherein the pin is configured to be received in a receptacle in the blunt end of a needle shaft and the shoulder is configured to engage the tip body.

In another aspect, the tip body may comprise a hollow tube which removably receives a tissue-penetrating distal end of the needle shaft so that the tissue-penetrating distal end projects distally from the distal end of the tip body to enable advancement of the needle shaft and tip body through tissue.

In other specific aspects of the present invention, the retention elements may formed of a plastic material. Alternatively, the retention elements may be formed of an elastic material. The retention elements may comprise barbs distributed axially along a proximally extending tail. Alternatively, the retention elements may comprise two or more resilient arms swept back from the tip body. Still further, the tip body may comprise a hollow cylinder adapted to be placed over a distal end of a needle shaft so that a proximal end of the cylinder engages a shoulder on the needle shaft. The retention elements may then be formed as cut-outs in the wall of the cylinder and be biased to project radially outwardly from the cylinder. The retention elements may also be formed as arms swept back from a proximal end of the cylinder.

In a second embodiment, a method for anchoring a length of suture in a tissue comprises advancing a needle shaft carrying an anchor tip body at its distal end through soft tissue. A length of suture is attached to the anchor tip, and the needle shaft may be retracted after the anchor tip body has reached a target location in the tissue. Such retraction causes the shaft to separate from the tissue leaving the tip body and suture in place. Retention elements on the anchor tip body deploy radially outwardly into the tissue to anchor the tip body in the tissue.

### BRIEF DESCRIPTION OF THE DRAWINGS

FIGS. 1A and 1B show side and isometric views of a T-bar anchor constructed in accordance with the principles of the present invention.

FIGS. 2A and 2B show side and isometric views of a grappling hook anchor constructed in accordance with the principles of the present invention.

FIGS. 3A and 3B show side and isometric views of a grappling hook tube anchor constructed in accordance with the principles of the present invention.

FIGS. 4A and 4B show side and isometric views of a barbed tube anchor constructed in accordance with the principles of the present invention.

### DETAILED DESCRIPTION OF THE FIRST EMBODIMENT

The anchors of the present invention may be fabricated from a variety of biocompatible polymers, metals, and combinations thereof. Suitable polymers or plastic materials include polypropylene, polydioxanone (PDO), poly(lactic-co-glycolic acid) (PGLA), polyglycolide co-caprolactone (PGCL), nylon, polyester, ultra high molecular weight polyethylene, or the like. Suitable, non-plastic materials include metals such as nitinol, stainless steel, titanium, or the like. It may be beneficial to have the anchor be made of the same material as the suture material.

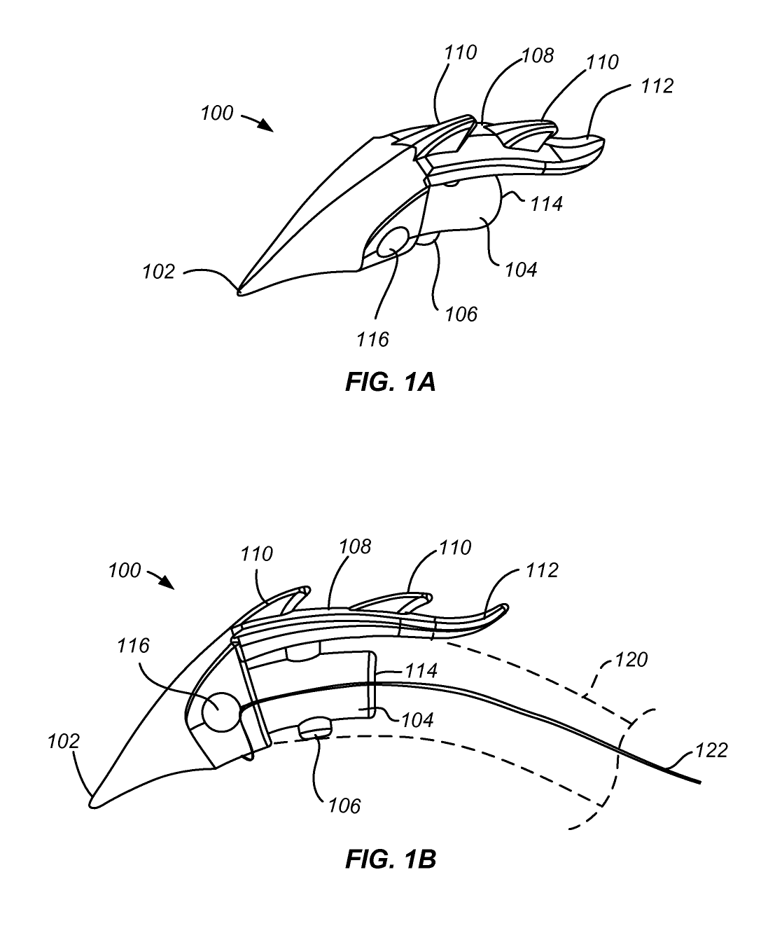

A first exemplary embodiment of a soft tissue suture anchor 100 is shown in FIGS. 1A and 1B. The anchor 100 is a T-bar having a distal guiding tip 102 which is tapered to allow tissue penetration so that the anchor 100 may be removably placed on a distal end of a needle shaft 120 (shown in broken line in FIG. 1A) which is used to advance the anchor 100 to penetrate the tissue and pull suture 122 distally through the tissue. A primary retaining peg 104 with one, two, or more secondary retaining features, such as grooves, bumps, or additional pegs 106, is located proximal to the distal guiding tip 102 and provide a “snap fit” for connecting the anchor to a receptacle in a distal end of the needle shaft 120. The anchor 100 has a proximal tail 108 which clips over the needle shaft 120 to help hold the anchor in place as the assembly of the needle shaft and anchor are advanced through tissue. Alternately or additionally, the anchor 100 may be retained on the needle shaft 120 by an interference fit or some other passive interface.

The proximal tail 108 also carries at least one and usually two or more barbs or fins 110 and a proximal lip 112. The barbs provide the desired anchoring in the tissue and prevent the anchor from being displaced in a proximal direction after it has been deployed. The proximal lip 112 allows the needle shaft to be rotated and detached from the anchor 100 after the anchor has been advanced to the desired target. The anchor 100 has a profile such that when rotated approximately 90 degrees after deployment, the underside of the proximal tail 108 serves as a T-bar which increases the contact area where the suture is pressing against tissue. If the suture does not rotate, the retention fins are intended to attach to the tissue. Should the anchor pass fully through a tissue volume into a space beyond a tissue wall, the entire anchor may serve as a pledget or T-bar type retention device. In the illustrated configuration, the suture 122 may be attached to a proximal surface 114 of the retaining peg of the anchor. Alternatively or additionally, the anchor may be connected to the suture in other ways such as by over-molding, crimping, use of a heat shrink material, passing through an opening 116 near a distal end of the anchor, or the like.

In use, the anchor 100 is placed in on a delivery device, such as the illustrated needle shaft 120, which is placed over the proximal peg 104 of the anchor with the tissue penetrating distal end of the anchor being the leading point for tissue entry. Alternately, a portion of the needle shaft may extend into or through a receptacle formed in a proximal end or surface of a tip body of the anchor 100. In the configuration shown, the proximal peg 104 has an additional perpendicular peg 106 which may snap into a channel of the needle shaft 120 to retain the anchor on the shaft and also aids in maintaining the proper orientation of the anchor. The needle shaft is used to guide the anchor through the tissue in any pattern, such as straight, circular, helical, or the like. The force of the retention of the anchor on the needle shaft is on the order of 0.1-10 Newtons. The anchor 100 is advanced to the proper position either within or through the relevant wall or layer of tissue/tissues. To deploy the anchor 100, the needle shaft 120 is retracted back through the tissue, and the barbs 110 on the anchor cause the anchor to stay in place. In an alternate embodiments (not illustrated), a direct or positive deployment mechanism may be used to release and position the anchor. For example, a wire running coaxially through the needle shaft may be extended at the end of advancing the anchor, thus ejecting the anchor from the needle shaft. Once the needle shaft is removed, the suture is tensioned to the desired force and a pledget may be used to maintain tension and provide a seal at the proximal end of the suture (not shown).

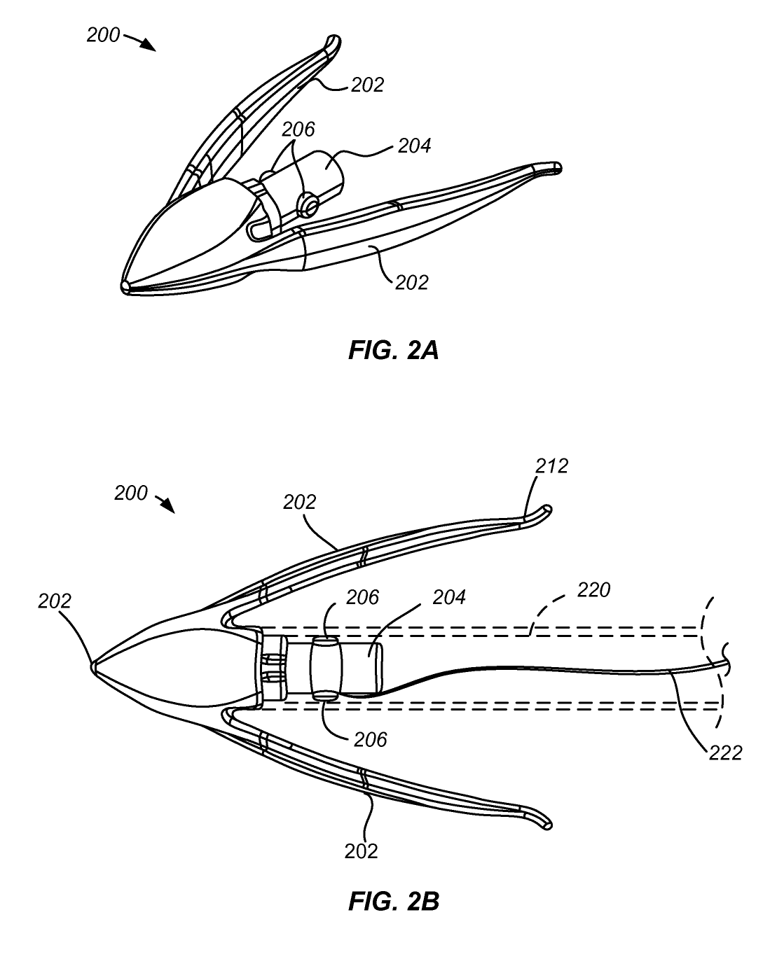

FIGS. 2A and 2B show an anchor 200 having a “grappling hook” design including a plurality of barbs or retention fins 202 (with two being illustrated) oriented radially around the body of the anchor provide the main source of attachment to tissue. The anchor 200 has a tip body with a tissue penetrating distal tip 202 and a primary retaining peg 204 which connects to a needle shaft 220 or other delivery mechanism as well as providing for connection to suture 222. The retention arms 202 project from the main body of the anchor and flare or sweep out such that they may flex and dig into the tissue when the anchor is tensioned proximally in tissue. This allows the anchor to have a narrow profile during insertion. In a specific embodiment, this anchor is made of a flexible plastic material, though non-plastic materials may also be used such as nitinol, stainless steel, titanium, or the like. Each retention arm 202 terminates in a proximal lip 212 which acts as described previously with respect to lips 112 in FIGS. 1A and 1B.

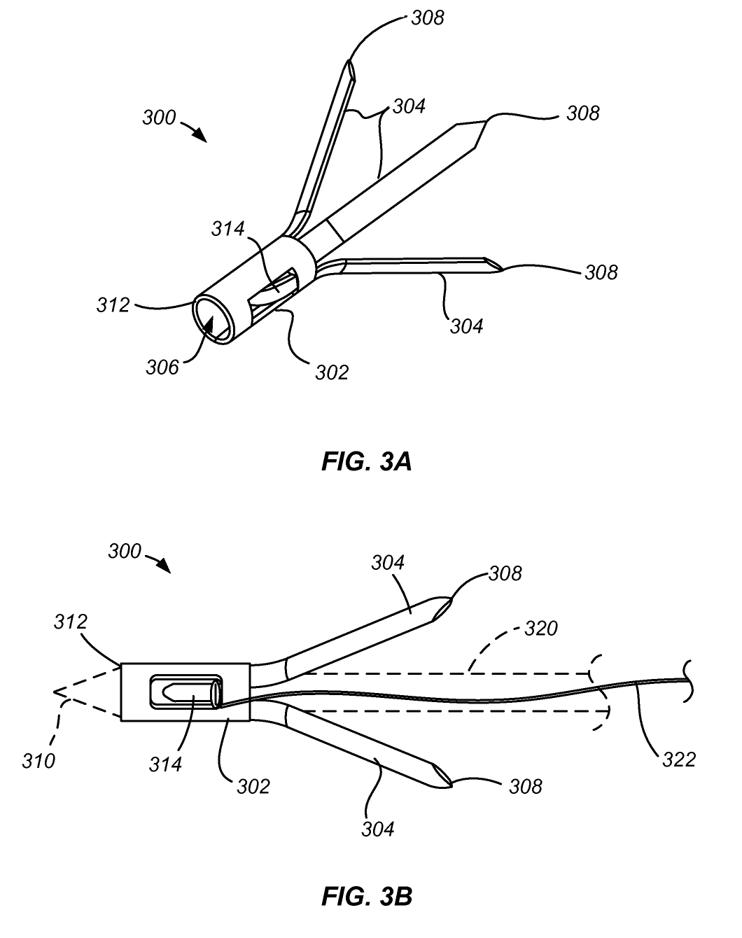

FIGS. 3A and 3B show a first variant of the “grappling hook” design where an anchor 300 comprises a tubular or cylindrical tip body 302 with a multitude of retention arms 304 distributed circumferentially around the tip body to provide the main source of attachment to tissue. Each retention arm 304 has a piercing tip 308 at its free or unsupported end (which is oriented proximally with respect to the tip body) so that the arm will penetrate into tissue as the anchor is urged in a proximal direction when tension is applied to suture 322. The cylindrical tip body 302 has a needle shaft lumen 306 which removably receives a needle shaft 320, and the needle shaft has a sharpened or other tissue penetrating tip 310 which projects distally from a distal end of the cylindrical tip body 302, allowing the user to puncture and guide the anchor 300 into and through tissue using the tip of the needle shaft and permitting the anchor to have a blunt tip 312. The suture may be attached to the cylindrical tip body 302 using a suture clip 314 cut out or otherwise formed in or on a wall of the cylindrical tip body 302.

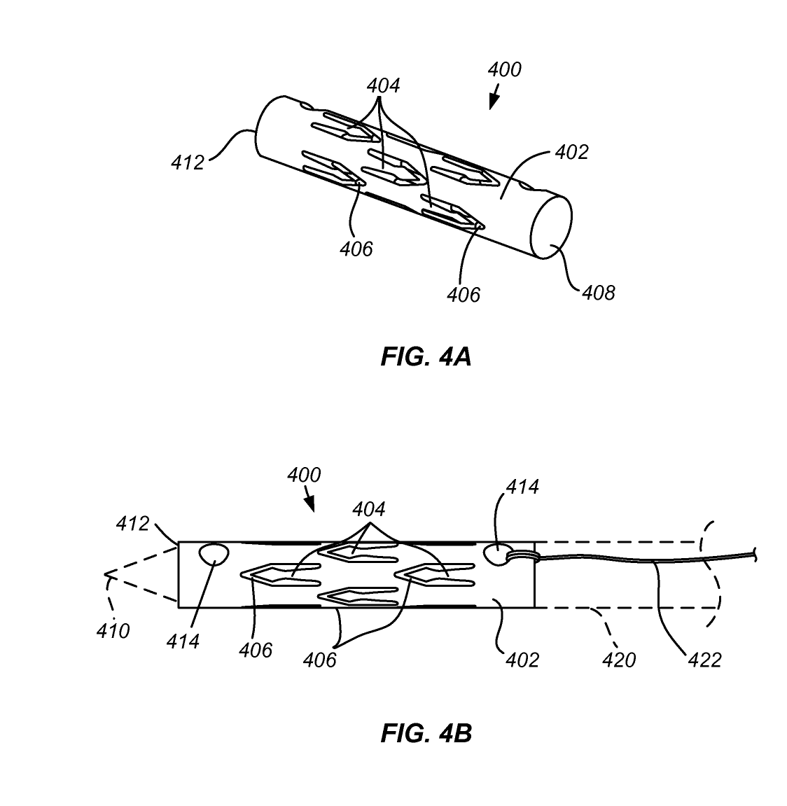

FIGS. 4A and 4B show a third variant of the “grappling hook” design where an anchor 400 comprises a tubular or cylindrical tip body 402 with a multitude of retention arms 404 distributed circumferentially and axially over the tip body to provide attachment to tissue. Each retention arm 404 has a piercing tip 406 at its free or unsupported end (which is oriented proximally with respect to the tip body) so that the arm will penetrate into tissue as the anchor is urged in a proximal direction when tension is applied to suture 422. The cylindrical tip body 402 has a needle shaft lumen 408 which removably receives a needle shaft 420, and the needle shaft has a sharpened or other tissue penetrating tip 310 which projects distally from a distal end of the cylindrical tip body 402, allowing the user to puncture and guide the anchor 400 into and through tissue using the tip of the needle shaft and permitting the anchor to have a blunt tip 312. The suture may be attached to the cylindrical tip body 302 through an aperture 414 cut out or otherwise formed in or on a wall of the cylindrical tip body 302. The aperture may be formed at the proximal end, at the distal end, or at multiple locations along the tip body 402.

The anchor 300 shown in FIG. 3 has fewer but larger retention arms 304, while the anchor 400 shown in FIG. 4 has more but smaller retention arms 404 which are formed as barbs. In FIGS. 4A and 4B, the barb-like retention arms 402 are shown in a constrained configuration lying within the space defined by the wall of the cylindrical tubular tip body. In use, the arms 404 will be biased radially outwardly so that they will penetrate into tissue when proximal tension is applied to the suture 422. The benefit of either may depend on the application. Suture ports 404 allow for the suture to be fed through and tied to the anchor, though the anchor may also be crimped on. In specific embodiments, to anchors 300 and 400 will be made of a super elastic material such as nitinol (nickel-titanium alloy), though other materials may also be used such as plastics, stainless steel, titanium, or the like.

The suture anchors of the present invention will find use whenever when a distal end of the suture attached to the anchor is to be left in place in solid tissue so that it cannot be tied off. They will find specific use with helical hollow needle suture deliver systems such as those described in commonly owned, co-pending U.S. patent application Nos. 13/169,454, filed Jun. 27, 2011; 13/224,666, filed Sep. 2, 2011; and 13/273,000, filed Oct. 13, 2011, the full disclosures of which are incorporated herein by reference

Although embodiments of various methods and devices are described herein in detail with reference to certain variation, it should be appreciated that other versions, embodiments, methods of use, and combinations thereof are also possible. Therefore, the scope of the appended claims should not be limited to the description of the embodiments contained herein.

What is claimed is:

1. An anchor for attaching to a distal end of a length of suture, said anchor comprising:
a tip body having a distal end and a proximal end;
at least one retention element projecting laterally from the tip body, said retention element being configured to permit the tip body to be advanced in a distal direction through tissue and to prevent the tip body from being withdrawn in a proximal direction from the tissue;
a suture attachment element on the tip body; and
a needle shaft engagement surface on the tip body, said needle shaft engagement surface being configured to be engaged by a distal end of a needle shaft to permit the needle shaft to push the tip body in a distal direction and to disengage from the tip body when drawn in a proximal direction.

2. An anchor as in claim 1, wherein the tip body has a tissue-penetrating distal end and wherein the needle shaft engagement surface is configured to engage a blunt end of the needle shaft.

3. An anchor as in claim 2, wherein the tip body has a pin surrounded by a shoulder at its proximal end, wherein the pin is configured to be received in a receptacle in a blunt end of a needle shaft and the shoulder is configured to engage the tip body.

4. An anchor as in claim 1, wherein the tip body comprises a hollow tube and is configured to removably receive a tissue-penetrating distal end of the needle shaft so that the tissue-penetrating distal end projects distally from the distal end of the tip body to enable advancement of the needle shaft and tip body through tissue.

5. An anchor as in claim 1, wherein the retention elements are formed of a plastic material.

6. An anchor as in claim 1, wherein the retention elements are formed of an elastic material.

7. An anchor as in claim 1, wherein the retention elements comprise barbs distributed axially along a proximally extending tail.

8. An anchor as in claim 1, wherein the retention elements comprise two or more resilient arms swept back from the tip body.

9. An anchor as in claim 1, wherein the tip body comprises a hollow cylinder adapted to be placed over a distal end of a needle shaft so that a proximal end of the cylinder engages a shoulder on the needle shaft.

10. An anchor as in claim 9, wherein the retention elements are formed as cut-outs in the wall of the cylinder and are biased to project radially outwardly from the cylinder.

11. An anchor as in claim 9, wherein the retention elements are formed as arms swept back from a proximal end of the cylinder.

12. A method for anchoring a length of suture in a tissue, said method comprising:
advancing a needle shaft carrying an anchor tip body at its distal end through soft tissue, wherein a length of suture is attached to the anchor tip;
retracting the needle shaft after the anchor tip body has reached a target location in the tissue, wherein the shaft separates from the tissue leaving the tip body and suture in place;
wherein retention elements on the anchor tip body deploy radially outwardly into the tissue to anchor the tip body in the tissue.
